(19) United States
(12) Patent Application Publication
(10) Pub. No.: US 2005/0021058 A1
(43) Pub. Date: Jan. 27, 2005

(54) Complete and universal implant for front path hernia repair

(76) Inventor: Paolo Negro, Roma (IT)

Correspondence Address: Oliff & Berridge, PO Box 19928, Alexandria, VA, 22320, US

(21) Appl. No.: 10/489,162

(22) PCT Filed: Sep. 20, 2002
(86) PCT No.: PCT/FR02/03226

(30) Foreign Application Priority Data
Sep. 21, 2001 (FR) 0112228

(51) Int. Cl. A61B 17/08

(52) U.S. Cl. 606/151

(57) ABSTRACT

The invention concerns an implant comprising: a planar part made of biocompatible material, knitted with monofilament or multifilament yarns with an armour providing it with run-resistant and macroporous properties; and a hollow protrusion made of biocompatible material, projecting from a surface of said planar part, said protrusion having a longitudinal axis perpendicular to the plane of the planar part, an orifice located in said plan and a flat base parallel to the planar part. The invention is characterized in that the protrusion is shaped like a cylindrical cup, and the base of said protrusion is spaced apart from said opening by a distance whereof the value is not more than the value of the diameter of the protrusion.

The present invention concerns an implant for treatment of hernias, in particular inguinal, femoral or crural hernias, by an anterior approach. It also concerns a method for production of this implant.

It is known to treat an inguinal, femoral or crural hernia by using, in combination, a flat implant in the form of a sheet, commonly referred to as a patch, and a three-dimensional implant forming an obturator and commonly referred to as a plug. The patch permits reinforcement of the posterior wall of the inguinal canal and helps avoid the risk of recurrence. The plug is introduced deep into the defect which is left by the reduction of the hernia and which it is able to close off.

It is also known, from document WO 97/35533, to form an implant comprising two layers made of different materials, one of which promotes adhesion of the tissues, and the other of which prevents such adhesion. By suitable folding of the implant, it is possible to exploit the respective properties of these layers. According to one embodiment described by said document, the implant comprises an expandable protuberance of frustoconical shape formed in the adhering layer.

This flexible and frustoconical protuberance does not seem able to adapt perfectly to the defect left by the reduction of the hernia, nor does it seem able to completely withstand the pressure exerted on it by the hernial sac once the implant is in place. The result of this is a not inconsiderable risk of recurrence, especially in the case of eversion of the protuberance under said pressure.

Admittedly, said document discloses the possibility of filling the cavity delimited by this protuberance and by the non-adhering layer, using an inert gas or another suitable inert material, and this seems able to provide the protuberance with a certain degree of rigidity.

However, this filling technique is considered to involve a number of serious operational drawbacks and is overall regarded as undesirable. Moreover, the resulting stiffening of the protuberance is temporary anyway, since the prior art document indicates that this protuberance, when it has been thus filled, can compress over the course of time as a result of the contraction of the tissues surrounding the hernial defect.

The object of the invention is to remedy all of these disadvantages.

The implant according to the invention comprises, in a manner known per se:
a flat part made of biocompatible material, knitted from monofilaments or multifilaments, with a structure which prevents unraveling and provides macroporosity, and
a hollow protuberance made of biocompatible material protruding from one face of this flat part, this protuberance having a longitudinal axis perpendicular to the plane of the flat part, an opening situated in this plane, and a flat base parallel to the flat part.

According to the invention, the protuberance is in the form of a cylindrical cell, and the base of this protuberance is spaced apart from said opening by a distance (h) whose value is not greater than the value of the diameter (d) of the protuberance.

The implant according to the invention thus specifically comprises a protuberance of cylindrical shape and with a height smaller than its diameter. This protuberance consequently has a peripheral wall perpendicular to said base and has a relatively limited height.

This shape gives the protuberance relative inherent rigidity, allowing it to completely withstand the pressure which the hernial sac exerts against said base once the implant is in place. Effective prevention of the risk of eversion is thus achieved.

This same shape allows the protuberance to adapt relatively precisely to the shape of the hernial defect, so that said peripheral wall comes into contact with the surrounding tissues which support this wall and thus contribute to the rigidity of the protuberance.

After it has been put in place, the protuberance can be fixed to these surrounding tissues by means of one or more sutures; the flat part can also be fixed to the surrounding tissues, in particular to the inguinal ligaments or to the inguinal floor, by one or more sutures.

The protuberance is preferably formed substantially at the middle of the width of the flat part and approximately one third of the way along the length of said flat part.

The flat part can thus be cut out as a function of the size of the dissection performed, so as to perfectly match the posterior wall of the inguinal canal. This cut is made without fraying and with emission of a minimum quantity of particles, by virtue of the non-unraveling structure constituting the knit.

In one embodiment of the invention, the protuberance has a diameter of the order of 20 millimeters and a height not greater than that diameter.

The flat part can be rectangular with straight or rounded corners. It can have a width of the order of 60 millimeters and a length of the order of 120 millimeters.

By virtue of its configuration and its dimensions, this implant is suitable for all types of hernial repairs, whether indirect, direct or crural.

In one embodiment, the face of the flat part intended to come into contact with the posterior anatomical structures has a roughness resulting from the texture and/or the structure of the knit from which this flat part is composed.

The protuberance can be formed independently of the flat part. The flat part then comprises a cutout formed in it in the zone of implantation of the protuberance, and the protuberance comprises a circular base joined by any means, such as welding, adhesive bonding or sewing, to the circular edge delimiting the cutout made in the flat part.

The flat part and the protuberance can then be made of knits which are different in terms of their texture and/or structure and which provide the properties sought for this flat part and for this protuberance, in particular a roughness or softness, and semi-rigidity or flexibility, respectively.

The flat part and/or the protuberance can be impregnated with substances which are bactericidal and/or anti-inflammatory and/or analgesic.

The flat part can also have, locally, a slit for the passage of the spermatic cord. The invention also relates to a method for obtaining the implant described above.

The method comprises the steps of:
a) producing a knit of thermoformable biocompatible material, consisting of monofilaments or multifilaments, and
b) stamping the knit so as to obtain the shape of the protuberance to be formed, and heating this knit, it being possible for this stamping operation and this heating to be done simultaneously or in succession.

The knit produced in step a) advantageously has dimensions such that, after shaping of the protuberance, it is also able to form said flat part, either as it stands or after appropriate cutting.

If not, the method comprises the steps of:
making a cut in a knit portion which, either as it stands or after appropriate cutting, is able to form said flat part, and
joining the protuberance by any means such as welding, adhesive bonding or sewing to the circular edge delimiting the cutout formed in the flat part.

The method can comprise, between steps a) and b) cited above, the steps of:
placing the knit between two plates made of heat-conducting material, the first of which plates comprises a hole with a diameter greater than the external diameter of the protuberance to be formed, and the second of which plates comprises a plug with a diameter slightly smaller than the internal diameter of the protuberance to be formed;
bringing these two plates together so that the plug of the second plate is engaged in the hole of the first plate, thereby effecting said stamping operation, and
keeping these plates in this position and heating them for the period of time necessary to form the protuberance.

Alternately, said second plate can comprise, instead of said plug, a hole with a diameter at least equal to the internal diameter of the protuberance to be formed, and a heating mandrel is engaged through the holes in the two plates so as to form the protuberance.

When the protuberance is made from a knit permitting formation of the flat part, the movement of the two plates toward each other is preferably such that the knit can slide between the plates during formation of the protuberance by means of said plug or said heating mandrel.

This sliding makes it possible to retain a substantial density of material remaining in the area of the protuberance.

The invention will be clearly understood from the following description of two embodiments of the implant, this description being made with reference to the attached diagrammatic drawing. In this drawing:

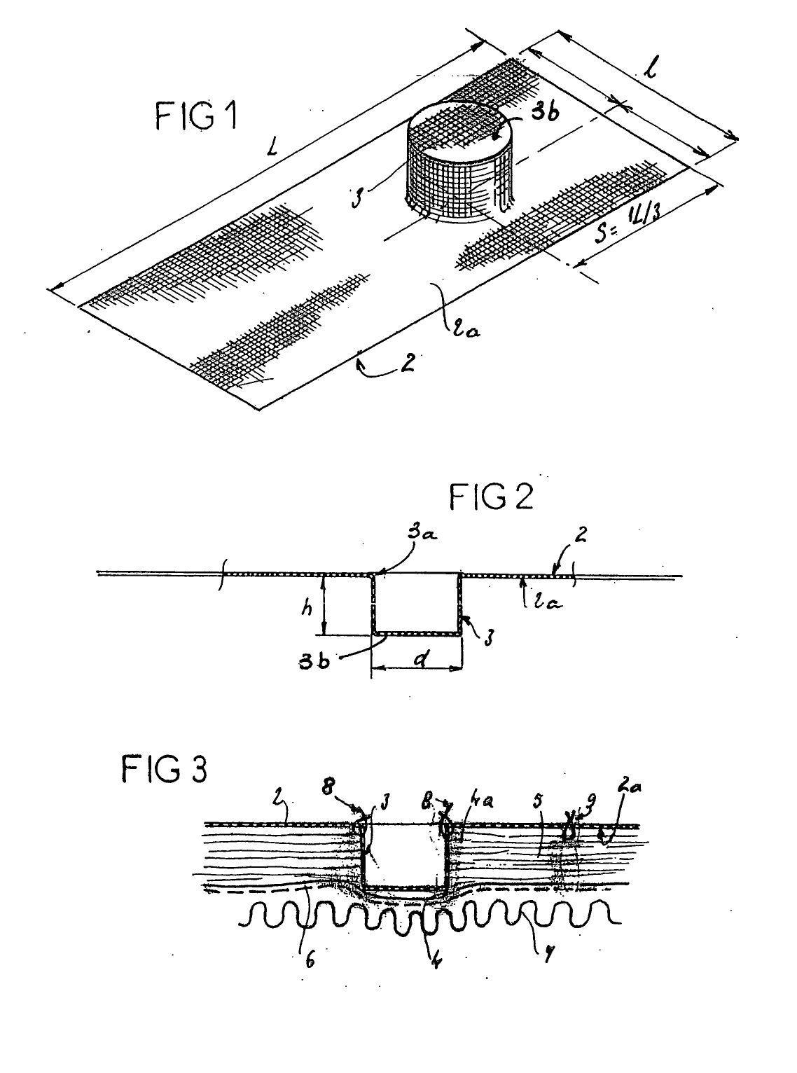

FIGS. 1 and 2 are views in perspective and in longitudinal section, respectively, of the implant according to a first embodiment;

FIG. 3 is a longitudinal section through this implant when it is in place in a hernial defect;

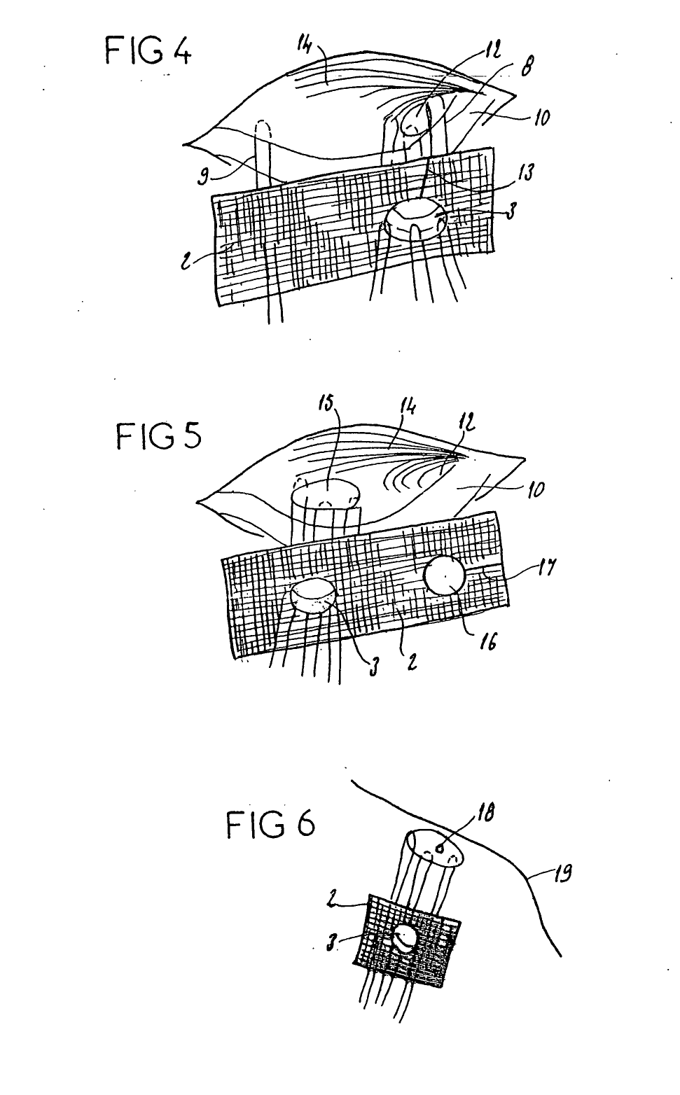

FIGS. 4, 5 and 6 are partial views of this implant, showing the conditions of placement of the implant for the repair of, respectively, a left indirect hernia, a left direct hernia, and a right crural hernia;

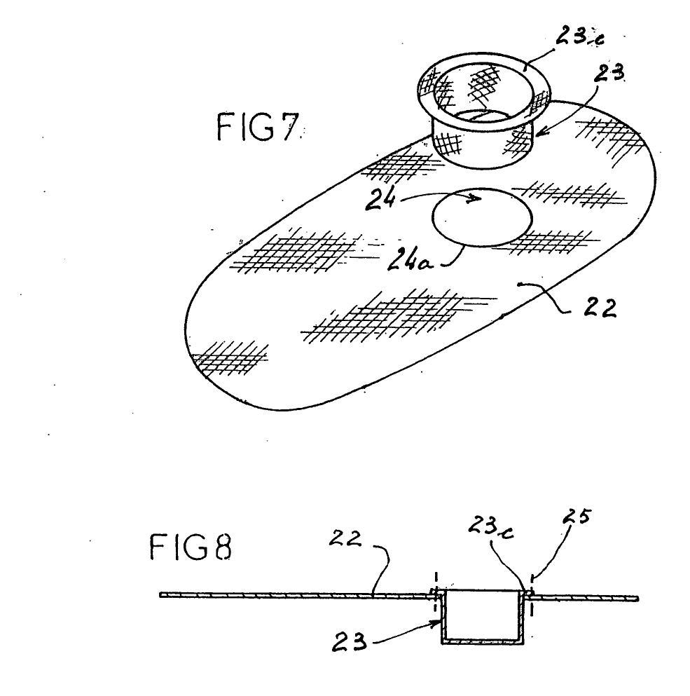

FIGS. 7 and 8 are views of the implant according to the second embodiment of the invention, FIG. 7 being a perspective view prior to assembly of its constituent elements, and FIG. 8 being a longitudinal section in the assembled state.

In the embodiment shown in FIGS. 1 and 2, the implant is composed of a flat part 2, of general rectangular shape, with straight or rounded corners. Protruding from one of the faces of this flat part 2 there is a protuberance 3 in the shape of a cylindrical cell whose longitudinal axis is perpendicular to the flat part 2. The opening 3a of the cell formed by the protuberance is situated in the plane of the flat part 2, and the base 3b of this protuberance 3, opposite the opening 3a, is flat and parallel to said flat part 2.

The flat part 2 and the protuberance 3 are in one piece and made from a knit of monofilaments or multifilaments of polypropylene or polyethylene having a diameter of between 0.10 and 0.20 millimeter.

According to a preferred embodiment of the invention, the implant is made from a knit of 0.15-mm polypropylene monofilaments, with open meshes, this knit being formed of two knitted sheets in accordance with the following respective configurations: sheet 1: 32/01/12/43; sheet 2: 32/01/12/43.

These structures form non-unraveling meshes which give the knit semi-rigidity, but with a possibility of deformation.

The cylindrical protuberance 3 is obtained by thermoforming of this knit.

According to one possible embodiment, the flat part 2 is compressed between two metal plates, of identical dimensions, one of which comprises a plug having the internal dimensions of the protuberance to be formed, and the other of which comprises an opening having the external diameter of the protuberance to be formed. The two plates are heated to a temperature of the order of 140° C. for about 25 seconds. The heat and the mechanical stress of engagement of the plug in the opening of the other plate, combined with the possibility of deformation of the knit constituting the flat part, form a protuberance in the latter, which protuberance at the end of production has relative rigidity and a cylindrical wall with an axis substantially perpendicular to the plane of the flat part.

The component thus formed is then subjected to cooling with air for a period of the order of 15 seconds.

The knit is not gripped tightly during the stamping operation and is therefore able to slide between the plates during production of the protuberance. This sliding makes it possible to retain a substantial density of material remaining in the area of the protuberance.

After the stamping operation, the component formed has the following characteristics:
density of the knit:
   in the area of the flat part: approximately 84 g/m²
   in the area of the base 3b: approximately 72 g/m²
   in the area of the peripheral wall of the protuberance 3: approximately 80 g/m²
rupture strength of the knit in the area of the flat part:
   longitudinal direction: 490 N
   transverse direction: 318 N
tear strength of the knit in the area of the flat part:
   longitudinal direction: 24 N
   transverse direction: 69 N
resistance to compression of the protuberance:
   1.7 N for a protuberance of approximately 25 mm in diameter and 18 mm in height
   2.6 N for a protuberance of approximately 25 mm in diameter and 14 mm in height.

According to another possible embodiment, the knit is placed between two metal plates heated to an adjustable temperature and comprising, facing one another, two cylindrical bores through which a heating mandrel can be engaged.

In a preferred embodiment, the flat part 2 is rectangular and has a width l of the order of 60 millimeters for a length L of the order of 120 millimeters, while the protuberance 3 has a diameter d of the order of 20 millimeters for a height h which is not greater than 20 millimeters and is, for example, 15 millimeters.

The protuberance 3 is formed so as to be substantially at the middle of the width of the flat part 2 and one third of the way along its length relative to one of its ends, as is shown by the distance S in FIG. 1.

The lower face 2a of the flat part 2, that is to say the face from which the protuberance 3 protrudes, can be rough so as to increase its ability to attach and adhere to the posterior anatomical structures. This roughness results from the texture and/or the structure used.

In the embodiment shown in FIGS. 7 and 8, the flat part 22 and the protuberance 23 are produced separately. The flat part 22, of rectangular shape with rounded ends 22a, comprises a circular cutout 24 in the zone of implantation of the protuberance 23. The protuberance 23 is cut, after its formation by thermoforming, from another knit in order to present the cylindrical shape shown in FIG. 7 with, near its opening 23a, a circular base 23c, straight or with a flange. After engagement of the cell 23 into the cutout 24, it is joined to the flat part 22, depending on the nature of the materials from which these two elements are made, by welding, bonding or sewing the circular base 23c of the protuberance 23 to the circular edge 24a of the cutout 24, as is illustrated by the broken line 25 in FIG. 8.

The flat part 22 and the protuberance 23 can thus be formed from knits which differ in their texture and/or their structure so as to give, respectively, a roughness or softness, and semi-rigidity or flexibility. Thus, for example, the flat part 22 is made of multifilaments of polyethylene or polyester, giving it softness on contact, with a structure which does not unravel but confers flexibility and forms burrs favoring adherence, while the knit in which the protuberance 23 is formed is made of monofilaments of polypropylene with a structure conferring greater deformability and semi-rigidity after thermosetting.

The implant, irrespective of the way it is formed, is intended to permit hernial repair by an open anterior approach. When it is put in place, and as is shown in FIG. 3, the protuberance 3 has dimensions allowing it to adapt to all the hernial openings 4 formed in the muscles 5 juxtaposed to the transverse fascia 6 and to the peritoneum 7. The connection of the protuberance 3 to the edges 4a of the muscle opening 4 is effected by sutures 8.

The flat part 2, which extends around the opening 4 against the inguinal wall, strengthens the latter, thus avoiding recurrences. This flat part 2 is fixed by a few suture points 9.

When the implant is thus in place, the protuberance 3 fits perfectly into the muscle opening 4 without generating any reaction indicative of the presence of a foreign body. Its top is flat and atraumatic, as it does not extend beyond the thickness of the wall, and limits the risks of deep visceral lesions caused by erosion. Moreover, by virtue of the rigidity of its constituent material and of its formation by thermoforming, there is no risk of the protuberance 3 deforming or undergoing eversion.

FIG. 4 shows the placement of an implant according to the invention for a left indirect hernia. To facilitate engagement of the protuberance 3 in the inguinal ring 12 and to permit passage of the spermatic cord 10, the surgeon adapts the implant by forming a slit 13 in it in order to allow this cord 10 to pass through. In the drawing, reference number 14 designates the external oblique muscles, and reference numbers 8 and 9 designate the suture threads described with reference to FIG. 3.

The surgeon can adapt the outer shape of the flat part 2 to the size of the dissection, in order to perfectly match the latter.

FIG. 5 shows the placement of an implant according to the invention in the context of repairing a left direct hernia. This involves repairing an orifice 15 formed in Hesselbach's triangle, in a zone more remote from the inguinal ring 12. For this repair, the implant is turned through 180° relative to the position it occupies in FIG. 3, so that, after the protuberance 3 has been placed in the orifice 15, the larger portion of the flat part 2 extends in the direction of the inguinal ring 12. To permit the passage of the spermatic cord 10, the surgeon forms an opening 16 with a slit 17 in the flat part 2 and, if appropriate, adapts the outer shape of the flat part 2 to the shape of the dissection.

FIG. 6 shows the treatment of a right crural hernia, which necessitates closure of the femoral ring 18, arranged in proximity to the inguinal ligament 19, by means of the protuberance 3. In this case, the surgeon adapts the implant by cutting it to length so as to give it substantially the same length on either side of the protuberance 3.

It will be evident from the above that the implant according to the invention is universal because it can be applied in the repair of indirect hernias, direct hernias and crural hernias, and that, in all these applications, it ensures not only the closure of the hernial orifice but also the strengthening of the inguinal floor around this orifice, thereby reducing the risks of recurrence, but without forming a body whose tightness would be a source of problems for the patient.

1. A complete and universal implant for the repair of hernias by an anterior approach, composed of a sheet of biocompatible material, made of monofilaments or multifilaments, knitted with a structure which prevents unraveling and provides macroporosity, and of a hollow protuberance made of biocompatible material protruding from one face of this sheet with which it forms a one-piece assembly; the sheet is flat and extends over a surface allowing it to cover the posterior wall of the inguinal canal, and the protuberance is situated at the middle of the width of the sheet and approximately one third of the way along its length, said protuberance having a longitudinal axis perpendicular to the sheet, forming an opening situated in the plane of the sheet, and having a base which lies opposite the opening and is flat and parallel to the sheet;
said implant being characterized in that the protuberance is in the form of a cylindrical cell, and in that its base, lying opposite said opening, is spaced apart from the latter by a distance whose value is not greater than the value of the diameter of the cell.

2. The implant as claimed in claim 1, characterized in that the protuberance in the form of a cell has a diameter whose value is of the order of 20 millimeters and a height whose value is not greater than that dimension.

3. The implant as claimed in claim 1, characterized in that the flat sheet, of overall rectangular shape with straight or rounded ends, has a width of the order of 60 millimeters and a length of the order of 120 millimeters, so as to form, around the protuberance, forming an obturator after its introduction into the opening of a wall, a flat implant which strengthens said wall and is able to be connected to it by sutures arranged away from the opening.

4. The implant as claimed in claim 1, characterized in that the face of the sheet intended to come into contact with the posterior anatomical structures has a roughness resulting from the texture and/or the structure of the knit from which this sheet is composed.

5. The implant as claimed in claim 1, characterized in that the protuberance in the shape of a cell is produced by thermal deformation of the sheet whose texture and/or structure give it semi-rigidity.

6. The implant as claimed in claim 1, characterized in that the protuberance in the shape of a cell is produced independently of the sheet and its circular base is connected by welding, adhesive bonding or sewing to the circular edge of a cutout formed in the sheet in the zone of implantation of this protuberance.

7. The implant as claimed in claim 6, characterized in that the sheet and the protuberance are made from knits which are different in terms of their texture and/or structure and which provide, respectively, a roughness or softness, and semi-rigidity or flexibility.

8. The implant as claimed in claim 1, characterized in that its sheet and its protuberance are impregnated by substances which are bactericidal and anti-inflammatory or analgesic.

9. The implant as claimed in claim 1, characterized in that its sheet has, locally, a slit for the passage of the spermatic cord.

10. A method for obtaining the implant as claimed in claim 1, characterized in that it comprises the steps of:
compressing the flat sheet between two metal plates, of identical dimensions, one of which comprises a plug having the internal dimensions of the protuberance to be formed, and the other of which comprises an opening having the external diameter of the protuberance;
heating the two plates;
engaging said plug in the opening of the other plate in such a way as to form, in the sheet, a rigid protuberance with a substantially cylindrical wall which is substantially at right angles to the flat surface of the sheet.

11. A method for obtaining the implant as claimed in claim 1, characterized in that it comprises the steps of:
placing the flat sheet between two metal plates which comprise, facing one another, two cylindrical bores through each of which a heating plug can be passed;
heating this plug;
engaging said plug in the opening of the other plate in such a way as to form, in the sheet, a rigid protuberance with a substantially cylindrical wall which is substantially at right angles to the flat surface of the sheet.
